(19) United States
(12) Patent Application Publication
(10) Pub. No.: US 2018/0044365 A1
(43) Pub. Date: Feb. 15, 2018

(54) PROCESSES FOR PREPARING FLUOROKETOLIDES

(71) Applicant: CEMPRA PHARMACEUTICALS, INC., Chapel Hill, NC (US)

(72) Inventors: David Eugene PEREIRA, Apex, NC (US); Stephen E. SCHNEIDER, Raleigh, NC (US); Keshav DEO, Vadodara, Gujarat (IN)

(21) Appl. No.: 15/555,701

(22) PCT Filed: Mar. 5, 2016
(86) PCT No.: PCT/US16/21085

§ 371 Date: Sep. 5, 2017

Related U.S. Application Data
(60) Provisional application No. 62/129,305, filed on Mar. 6, 2015.

(51) Int. Cl. C07H 17/08

(52) U.S. Cl. CPC C07H 17/08

(57) ABSTRACT

Processes and intermediates for preparing fluoroketolide compounds are described herein.

### CROSS-REFERENCE TO RELATED APPLICATIONS

This application claims the benefit under 35 U.S.C. §119(e) of U.S. Provisional Application Ser. No. 62/129,305, filed Mar. 6, 2015, the disclosure of which is incorporated herein by reference.

### TECHNICAL FIELD

The invention described herein pertains to processes and intermediates for preparing fluoroketolide compounds.

### BACKGROUND AND SUMMARY OF THE INVENTION

Fluoroketolide compounds have been reported to be highly effective in treating bacterial and protozoal infections. Moreover, fluoroketolide compounds have been reported to be particular effective in treating resistant bacterial and protozoal infections compared to corresponding non-fluoroketolides, macrolides, and azalides. However, reported manufacturing processes for ketolides proceed with low conversion, which leads to sometimes insurmountable purification problems in separating the fluorinated product from the non-fluorinated starting material. In addition, reported manufacturing processes for ketolides tend to produce high amounts of unwanted side products, such as N-demethylated side products. Taken together, reported manufacturing processes for ketolides may be incapable of providing the needs of the world.

Due to the importance of these compounds for human and other animal health, alternative and/or improved processes for their preparation are needed. In particular, processes for preparing fluoroketolides at commercially relevant manufacturing scales are needed in order to satisfy the unmet need for these important human and animal health compounds.

It has been unexpectedly discovered herein that processes including amine bases provide high conversion rates to fluoroketolides, with fewer side products. Because of high conversion rates and fewer side products, processes described herein can be used to prepare multi-kilogram quantities of fluoroketlides, which may be isolated by simple precipitation, rather than by chromatography or fractional recrystallization, each of which are expensive and/or can lead to significant losses in isolated yield.

In one illustrative embodiment of the invention described herein, processes are described for preparing fluoroketolide compounds by fluorination at C2 of the macrocycle. In another embodiment, the processes described herein include the following step:

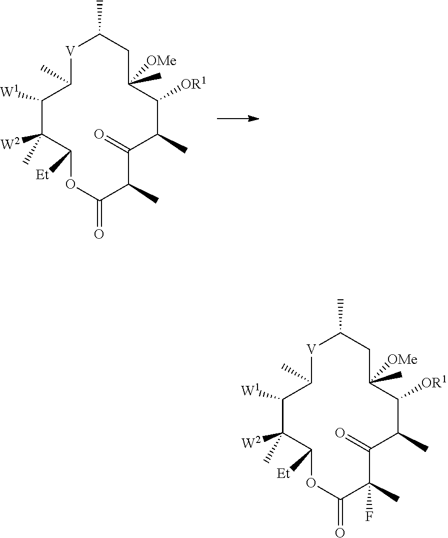

including salts of each of the foregoing, wherein:

R¹ is H or acyl, or R¹ is a monosaccharide, such as a methylamino or dimethylamino containing monosaccharide;

V is CH2—N(R), C═Q or C═NQ¹; where Q is O or (NR, H); where R is hydrogen or optionally substituted alkyl; and Q¹ is hydroxy or a derivative thereof or amino or a derivative thereof; and

W¹ is hydroxy or a derivative thereof; and W² is H, or hydroxy or a derivative thereof; or W¹ and W² are taken together with the attached carbon atoms to form an oxygen and/or nitrogen containing heterocycle, each of which is optionally substituted.

In another embodiment, the processes include the following step:

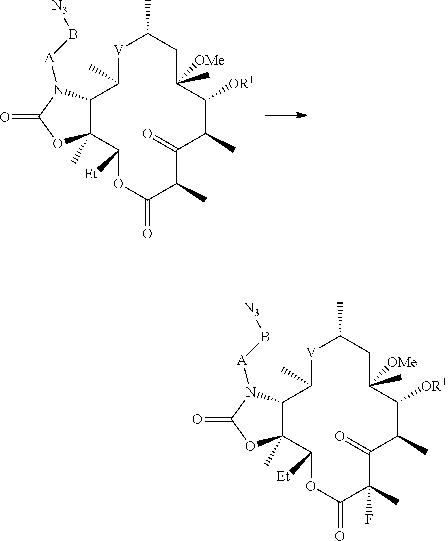

including salts of each of the foregoing, wherein:

R¹ is H or acyl, or R¹ is a monosaccharide, such as a methylamino or dimethylamino containing monosaccharide;

V is CH2—N(R), C═Q or C═NQ¹; where Q is O or (NR, H); where R is hydrogen or optionally substituted alkyl; and Q¹ is hydroxy or a derivative thereof or amino or a derivative thereof;

A is a bond, or A is an optional linker formed from O, C(O), CR, CR₂, and NR, and combinations thereof, where each R is independently selected in each instance from being absent to form a double or triple bond, being hydrogen, or being an optionally substituted alkyl; and

B is a bond, or B is an optionally substituted alkylene, optionally substituted alkenylene, or optionally substituted alkynylene.

In another embodiment, the processes include the following step:

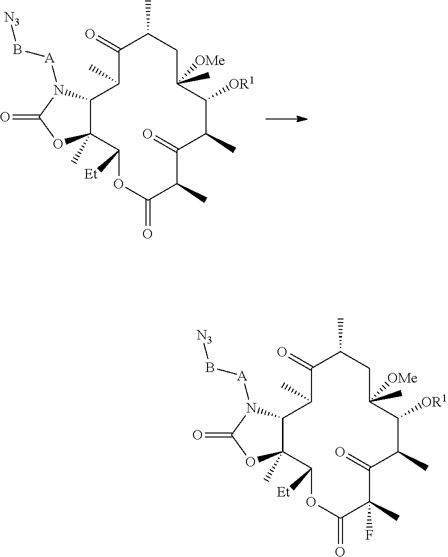

including salts of each of the foregoing, wherein:

R¹ is H or acyl, or R¹ is a monosaccharide, such as a methylamino or dimethylamino containing monosaccharide;

A is a bond, or A is an optional linker formed from O, C(O), CR, CR₂, and NR, and combinations thereof, where each R is independently selected in each instance from being absent to form a double or triple bond, being hydrogen, or being an optionally substituted alkyl; and

B is a bond, or B is an optionally substituted alkylene, optionally substituted alkenylene, or optionally substituted alkynylene.

In another embodiment, processes are described herein for preparing fluoroketolide compounds by in situ N-methylation. In another embodiment, the processes include the following step:

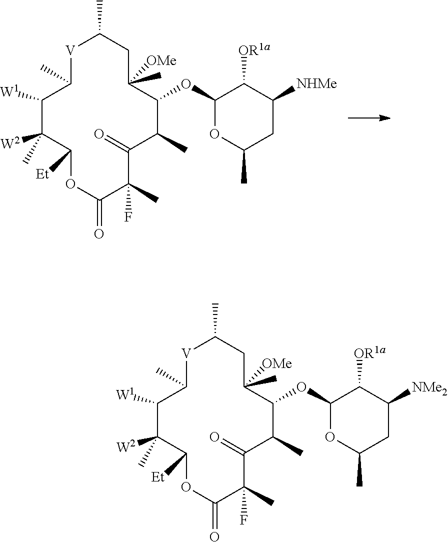

including salts of each of the foregoing, wherein:

R^{1a} is H or acyl;

V is CH2—N(R), C═Q or C═NQ¹; where Q is O or (NR, H); where R is hydrogen or optionally substituted alkyl; and Q¹ is hydroxy or a derivative thereof or amino or a derivative thereof;

W¹ is hydroxy or a derivative thereof; and W² is H, or hydroxy or a derivative thereof; or W¹ and W² are taken together with the attached carbon atoms to form an oxygen and/or nitrogen containing heterocycle, each of which is optionally substituted.

In another embodiment, the processes include the following step:

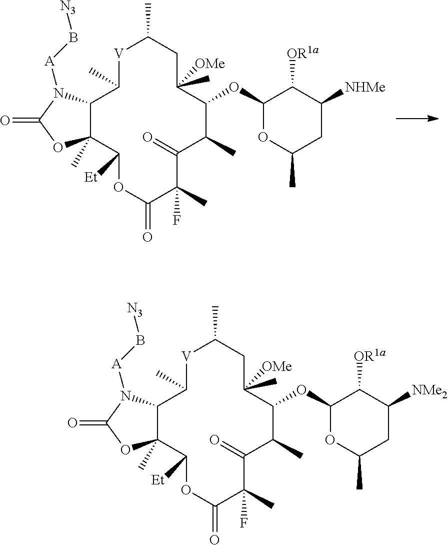

including salts of each of the foregoing, wherein:

R^{1a} is H or acyl;

V is CH2—N(R), C═Q or C═NQ¹; where Q is O or (NR, H); where R is hydrogen or optionally substituted alkyl; and Q¹ is hydroxy or a derivative thereof or amino or a derivative thereof;

A is a bond, or A is an optional linker formed from O, C(O), CR, CR₂, and NR, and combinations thereof, where each R is independently selected in each instance from being absent to form a double or triple bond, being hydrogen, or being an optionally substituted alkyl; and

B is a bond, or B is an optionally substituted alkylene, optionally substituted alkenylene, or optionally substituted alkynylene.

In another embodiment, the processes include the following step:

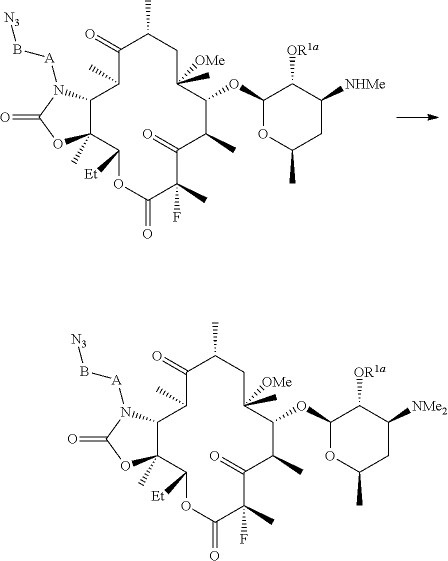

including salts of each of the foregoing, wherein:

R^{1a} is H or acyl;

A is a bond, or A is an optional linker formed from O, C(O), CR, CR₂, and NR, and combinations thereof, where each R is independently selected in each instance from being absent to form a double or triple bond, being hydrogen, or being an optionally substituted alkyl; and

B is a bond, or B is an optionally substituted alkylene, optionally substituted alkenylene, or optionally substituted alkynylene.

In another embodiment, the processes include the following step:

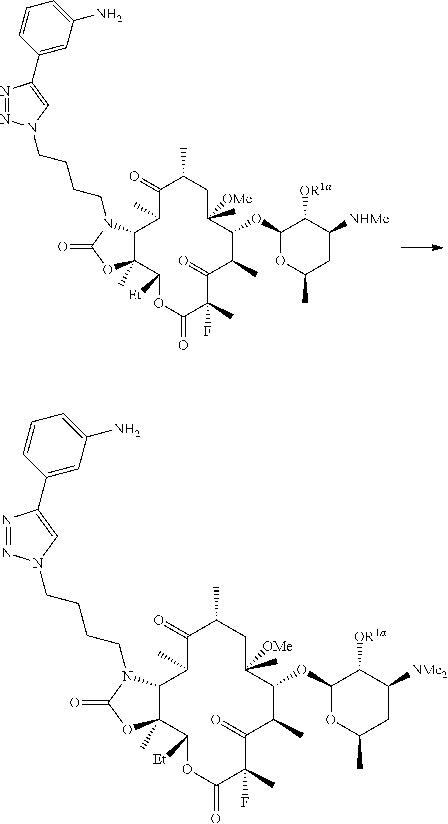

including salts of each of the foregoing, wherein R^{1a} is H or acyl.

In another embodiment, intermediates for preparing fluoroketolide compounds are described herein. Illustrative intermediates are of the formulae

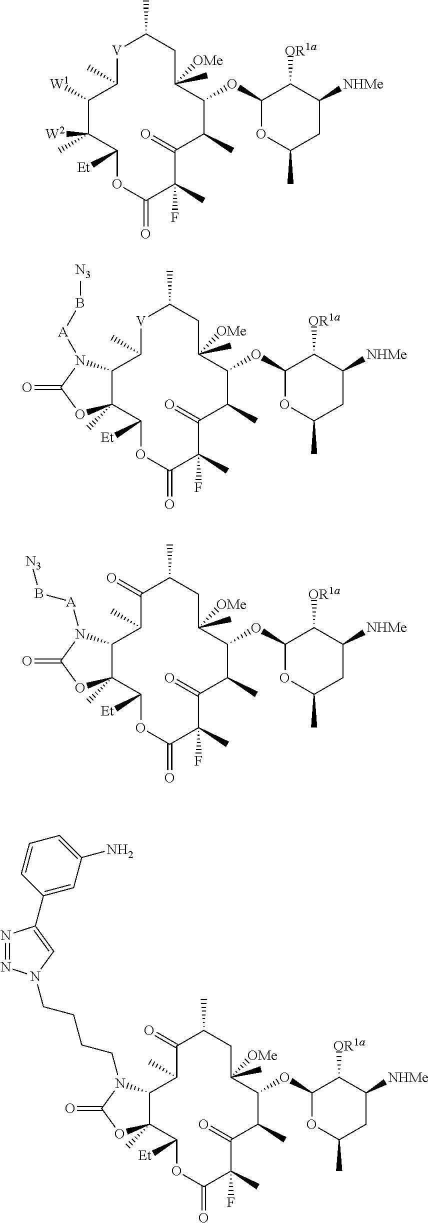

and salts thereof, wherein R^{1a}, V, W¹, W², A, and B are as defined herein.

In another embodiment, pharmaceutical compositions containing one or more of the compounds are also described herein. It is to be understood that the compositions may include other components and/or ingredients, including, but not limited to, other therapeutically active compounds, and/or one or more carriers, diluents, excipients, and the like, and combinations thereof.

In another embodiment, methods for treating host animals with a bacterial or protozoal infection are also described herein, where the methods include administering one or more of the compounds and/or compositions described herein to the host animal. In another embodiment, uses of the compounds and compositions in the manufacture of a medicament for treating host animals with a bacterial or protozoal infection are also described herein. In another embodiment, the medicaments include a therapeutically effective amount of the one or more compounds and/or compositions for treating a host animal with a bacterial or protozoal infection.

### DETAILED DESCRIPTION

Certain fluoroketolides and processes for preparing fluoroketolides are described in WO 2004/080391. Processes for preparing fluoroketolides are also described in WO 2009/055557. It has been discovered that the processes described for fluorination at C2 of the macrolide core structure in the foregoing publications fail to go to completion. In addition, it has been discovered that attempts to effect complete fluorination at C2 leads to greater and greater amounts of side product formation, such as N-demethylation of sugars at C5, such as demethylation of desosamine, and decomposition as the reaction conditions become more vigorous. Thus, the quest to solve the problem of incomplete conversion, leads to at least two other problems, side product formation and lower overall yields. Moreover, it has also been unexpectedly discovered that the unfluorinated starting material and the fluorinated product are essentially inseparable, especially using commercially relevant purification techniques that are necessary for the large scales required to produce antibiotics for a worldwide market. Because the only difference between the starting material and the desired product is a single fluorine atom, separation of the two compounds is quite difficult, and can only be accomplished by careful column chromatography or fractional recrystallizations, which each result in substantial material loss, and consequentially, an overall loss in yield. It has also been unexpectedly discovered that the N-desmethyl side product is also very difficult to remove using commercially relevant purification techniques that are necessary for the large scales required to produce antibiotics for a worldwide market. Commercially relevant purification techniques include evaporations, precipitations, and crystallizations, whereas chromatography, or fractional crystallization, each of which is much more expensive and leads to substantial decreases in yield, are advantageously avoided.

In addition, it has been discovered that in many instances, the corresponding unfluorinated analog of the desired fluoroketolide is substantially less active than the desired fluorinated compound, especially against resistant pathogens. Similarly, it has been discovered that in nearly all instances the corresponding N-demethylated analog of the desired fluoroketolide is substantially less active than desired the N,N-dimethyl compound. Thus, it is understood that complete fluorination is desirable to ensure that the product is pure, and also, that it is not contaminated with less active analogs that might affect drug performance, especially when the relative amount of those less active analogs might vary from across multiple batches. Similarly, it is understood that averting demethylation is desirable to ensure that the product is pure, and also, that it is not contaminated with less active analogs that might affect drug performance, especially when the relative amount of those less active analogs might vary from across multiple batches.

Solving the problem of incomplete fluorination requires more vigorous reagents and reaction conditions, such as higher temperatures, more equivalents of base, and/or more equivalents of fluorinating agent. However, those same process modifications exacerbate the companion problem by increasing the amount of unwanted N-demethylated products, both of the starting unfluorinated compound, for example (1-DM), and the product fluorinated compound, for example (2-DM).

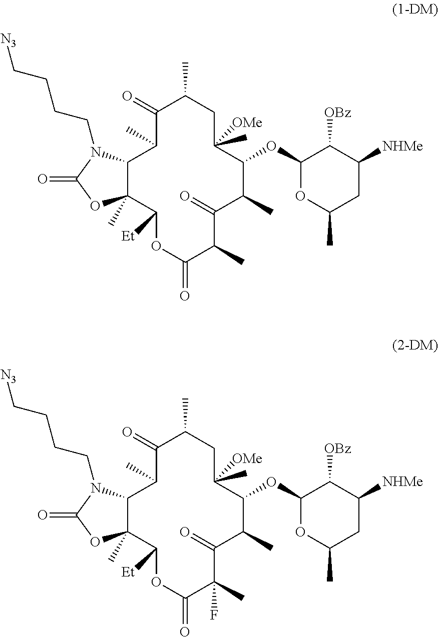

In addition, use of those same more vigorous reagents and reaction conditions leads to decomposition, other unwanted side products, and consequentially, an overall loss in yield.

Similarly, solving the problem of unwanted N-demethylation requires less vigorous reagents and reaction conditions. However, that same solution exacerbates the companion problem by decreasing the conversion of unfluorinated compound to the product fluorinated compound.

It has also been discovered that even when the process reported in WO 2009/055557 is modified to favor conversion, and concomitant N-demethylation, that in situ remethylation fails. Therefore, it is necessary to isolate the multiple products from the reaction mixture, and perform a separate remethylation step, which leads to additional material losses, an overall drop in yield, higher costs, and longer manufacturing times.

The need for fluoroketolides for treatment of bacterial and protozoal infections worldwide requires a manufacturing process that is both cost effective and can be performed on large scale. Without those attributes, supplies of fluoroketolides will be insufficient to meet the needs of world, and/or preclude the use of fluoroketolides in the poorer regions of the world, where bacterial or protozoal infections are often more prevalent, and lead to poorer outcomes.

New processes for preparing fluoroketolides are needed. Without such improved processes that provide higher yields of highly pure fluoroketolide antibiotics, there is a risk that millions of patients having bacterial or protozoal infections will go untreated due to short supply, delayed manufacturing and/or treatment costs that are too high.

It has been unexpectedly discovered herein that the fluorination processes described herein provide substantially higher conversion of unfluorinated starting material to the needed fluoroketolides. It has also been unexpectedly discovered herein that the fluorination processes described herein provide substantially lower amounts of N-demethylated side products. In addition, it has unexpectedly discovered herein that the fluorination processes described herein can be adapted to include in situ remethylation to further improve overall yields by recapturing N-demethylated side products. Therefore, the unwanted N-demethylation products, including for example (1-DM) and (2-DM), are useful as starting materials for preparing fluoroketolides. The processes described herein provide fluorinated ketolides in high yields, with high purity, and are adaptable to large multi-kilogram commercial manufacturing scales.

Several illustrative embodiments of the invention are described by the following clauses:

A process for preparing a fluoroketolide of formula (I)

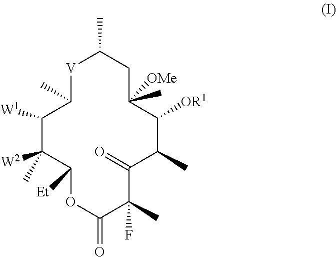

the process comprising the step of contacting a compound of the formula

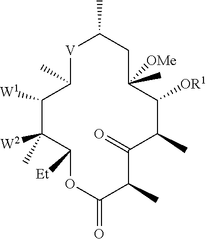

with a fluorinating agent and an amine base; wherein

R¹ is H or acyl, or R¹ is a monosaccharide, such as methylamino or dimethylamino containing monosaccharide;

V is CH2—N(R), C═Q or C═NQ¹; where Q is O or (NR, H); where R is hydrogen or optionally substituted alkyl; and Q¹ is hydroxy or a derivative thereof or amino or a derivative thereof; and

W¹ is hydroxy or a derivative thereof; and W² is H, or hydroxy or a derivative thereof; or W¹ and W² are taken together with the attached carbon atoms to form an oxygen and/or nitrogen containing heterocycle, each of which is optionally substituted.

Any one of the preceding processes wherein the amine base is a cyclic, a non-aromatic amine base, or a base with a conjugate acid pKa of at least about 11, at least about 11.5, at least about 12, at least about 12.5, or at least about 13, or a combination of the foregoing.

Any one of the preceding processes wherein the base is a cyclic amine.

Any one of the preceding processes wherein the base is a bicyclic amine.

Any one of the preceding processes wherein the base is sterically hindered.

Any one of the preceding processes wherein the base is conformationally restricted.

Any one of the preceding processes wherein the base is a diamine.

Any one of the preceding processes wherein the base includes at least one nitrogen that does not have a hydrogen.

Any one of the preceding processes wherein the base does not include any NH groups.

Any one of the preceding processes wherein the base includes at least one C=N group.

Any one of the preceding processes wherein the base is selected from the group consisting of 1,8-diazabicyclo[5.4.0]undec-7-ene (DBU), 1,5-diazabicyclo[4.3.0]non-5-ene (DBN), and 3,3,6,9,9-pentamethyl-2,10-diazabicyclo[4.4.0]dec-1-ene (PMDBD), quinuclidine, and combinations thereof.

Any one of the preceding processes wherein the base is DBN or DBU, or a combination thereof.

Any one of the preceding processes wherein the base is DBU.

Any one of the preceding processes wherein the fluorinating agent is selected from the group consisting of NFSi, Selectfluor, and F-TEDA, and combinations thereof.

Any one of the preceding processes wherein the fluorinating agent is selected from the group consisting of NFSi and Selectfluor, and combinations thereof.

Any one of the preceding processes wherein the fluorinating agent is a combination of NFSi and Selectfluor.

Any one of the preceding processes wherein the fluorinating agent is NFSi.

Any one of the preceding processes wherein the temperature is between about −30° C. and about −20° C. It has been observed that at temperatures closer to ambient temperatures, an increasing number of side products begin to form.

A process for preparing a compound of formula (I), the process comprising the step or further comprising the step of contacting a compound of the formula (DM)

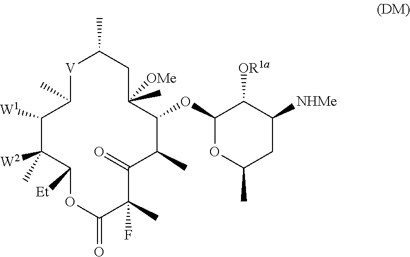

including salts of each of the foregoing, with a methylating agent; wherein:

R^{1a} is H or acyl;

V is CH2—N(R), C═Q or C═NQ¹; where Q is O or (NR, H); where R is hydrogen or optionally substituted alkyl; and Q¹ is hydroxy or a derivative thereof or amino or a derivative thereof;

W¹ is hydroxy or a derivative thereof; and W² is H, or hydroxy or a derivative thereof; or W¹ and W² are taken together with the attached carbon atoms to form an oxygen and/or nitrogen containing heterocycle, each of which is optionally substituted.

Any one of the preceding processes wherein the methylating agent is CH₂O/HCO₂H.

Any one of the preceding processes performed in a solvent wherein the solvent comprises a ketone, such as acetone, MEK, or MTBK.

Any one of the preceding processes performed in a solvent wherein the solvent comprises an ether, such as MTBE, THF, Me-THF, or a glycol ether, such as dimethoxyethane, diethoxyethane, or a compound of the formula R¹O—(CH₂)₂—OR², where R¹ is alkyl, such as methyl, ethyl, propyl, isopropyl, or butyl; and R² is H, methyl, ethyl, propyl, isopropyl, or butyl;

or a compound of the formula R¹[O—(CH₂)₂—]₂OR², where R¹ is alkyl, such as methyl, ethyl, propyl, isopropyl, or butyl; and R² is H, methyl, ethyl, propyl, isopropyl, or butyl.

Any one of the preceding processes performed in a solvent wherein the solvent comprises an ester, such as EtOAc, iPrOAc.

Any one of the preceding processes performed in a solvent wherein the solvent comprises an amide, such as DMF, DMA, NMP.

Any one of the preceding processes performed in a solvent wherein the solvent comprises a mixture of an amide and an ester, such as iPrOAc/DMF, or iPrOAc/DMF illustratively at a ratio in the range from about 1:2 to about 2:1, or about 3:2 to about 2:3, or about 1:1.

Any one of the preceding processes performed in a solvent wherein the solvent is substantially free of or free of chlorinated solvents, such as CH₂Cl₂ (DCM), CHCl₃, and/or CCl₄.

Any one of the preceding processes wherein W¹ and W² are taken together with the attached carbon atoms to form to carbamate where the nitrogen thereof is substituted with a radical of the formula N₃-B-A, where A is a bond, or A is an optional linker formed from O, C(O), CR, CR₂, and NR, and combinations thereof, where each R is independently selected in each instance from being absent to form a double or triple bond, being hydrogen, or being an optionally substituted alkyl; and B is a bond, or B is an optionally substituted alkylene, optionally substituted alkenylene, or optionally substituted alkynylene.

Any one of the preceding processes wherein W¹ and W² are taken together with the attached carbon atoms to form to carbamate where the nitrogen thereof is substituted with a radical of the formula T-B-A, where A is a bond, or A is an optional linker formed from O, C(O), CR, CR₂, and NR, and combinations thereof, where each R is independently selected in each instance from being absent to form a double or triple bond, being hydrogen, or being an optionally substituted alkyl; B is a bond, or B is an optionally substituted alkylene, optionally substituted alkenylene, or optionally substituted alkynylene; T is an optionally substituted aryl group, including but not limited to, imidazolyl, 1,2,3-triazolyl, phenyl, benzimidazolyl, benztriazolyl, and the like, and where the optional substitution, includes but is not limited to optionally substituted aryl, such as phenyl, aminophenyl, benzimidazolyl, benztriazolyl, benzimidazolylmethyl, benztriazolylmethyl, and the like.

Any one of the preceding processes wherein the compound of formula (I) is

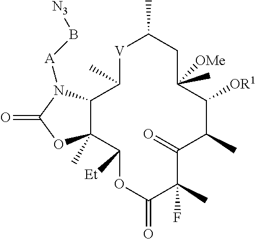

or a salt thereof.

Any one of the preceding processes wherein the compound of formula (I) is

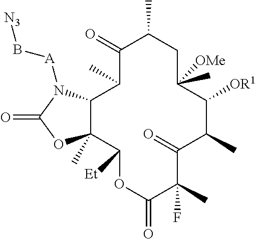

or a salt thereof.

Any one of the preceding processes wherein the compound of formula (I) is

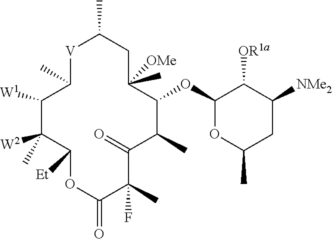

or a salt thereof.

Any one of the preceding processes wherein the compound of formula (I) is

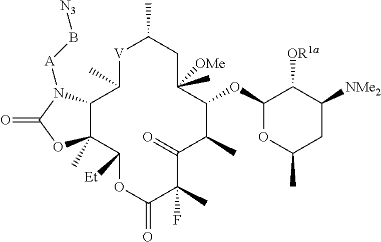

or a salt thereof.

Any one of the preceding processes wherein the compound of formula (I) is

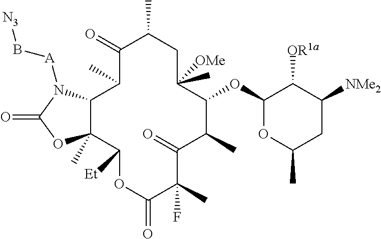

or a salt thereof.

Any one of the preceding processes wherein the compound of formula (I) is solithromycin or a salt thereof.

Any one of the preceding processes wherein the compound of formula (DM) is

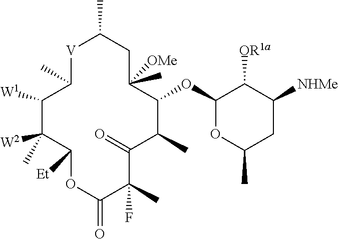

or a salt thereof.

Any one of the preceding processes wherein the compound of formula (DM) is

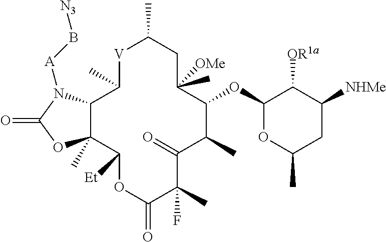

or a salt thereof.

Any one of the preceding processes wherein the compound of formula (DM) is

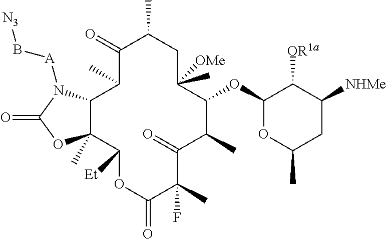

or a salt thereof.

Any one of the preceding processes wherein the starting compound is of the formula

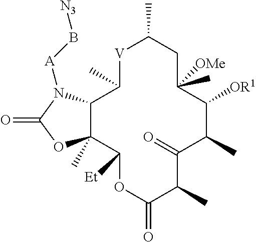

or a salt thereof.

Any one of the preceding processes wherein the starting compound is of the formula

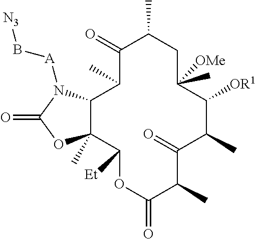

or a salt thereof.

Any one of the preceding processes wherein the starting compound is of the formula

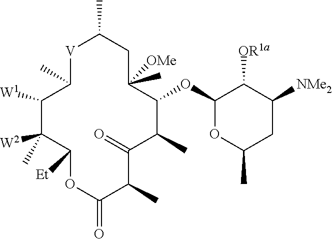

or a salt thereof.

Any one of the preceding processes wherein the starting compound is of the formula

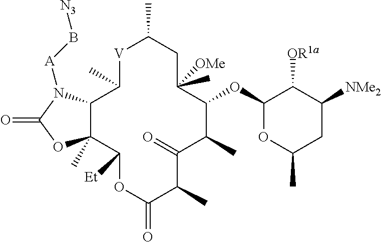

or a salt thereof.

Any one of the preceding processes wherein the starting compound is of the formula

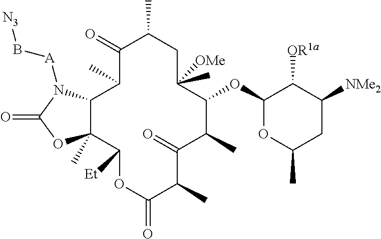

or a salt thereof.

Any one of the preceding processes wherein the starting compound is of the formula

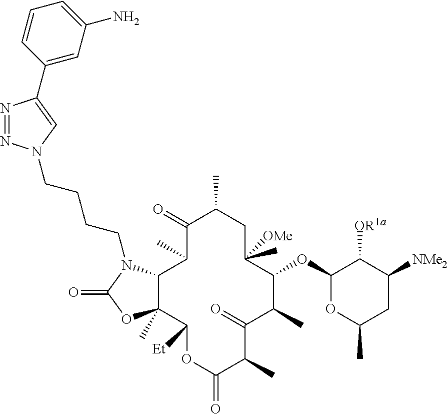

or a salt thereof.

Any one of the preceding processes wherein the starting compound is of the formula

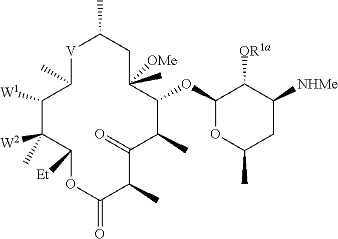

or a salt thereof, or the C2-fluoro analog of the foregoing.

Any one of the preceding processes wherein the starting compound is of the formula

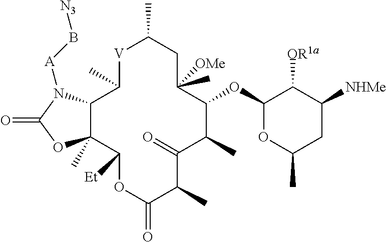

or a salt thereof, or the C2-fluoro analog of the foregoing.

Any one of the preceding processes wherein the starting compound is of the formula

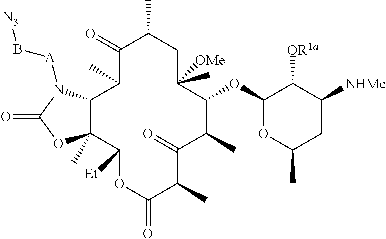

or a salt thereof, or the C2-fluoro analog of the foregoing.

Any one of the preceding processes wherein the starting compound is of the formula

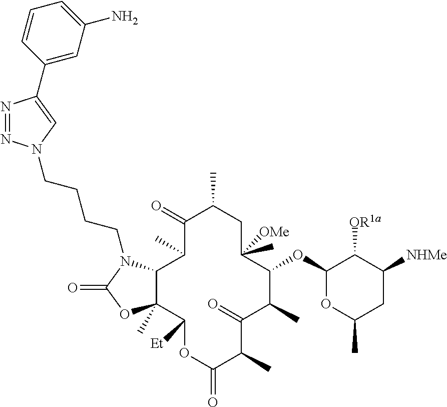

or a salt thereof, or the C2-fluoro analog of the foregoing.

Any one of the preceding processes wherein the compound is of the formula

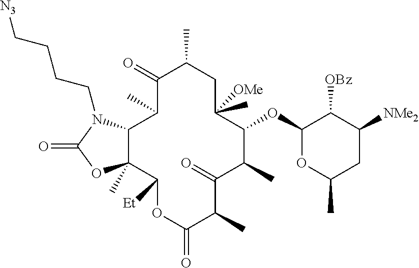

or a salt thereof.

Any one of the preceding processes wherein the compound is of the formula

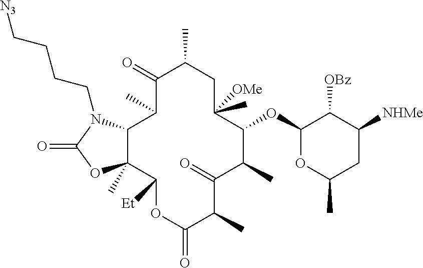

or a salt thereof.

Any one of the preceding processes wherein the monosaccharide is a hexose, such as D-glucose, D-mannose, D-xylose, D-galactose, L-fucose, and the like; a pentose such as D-ribose, D-arabinose, and the like; a ketose such as D-ribulose, D-fructose, and the like; including aminomethyl and dimethylamino derivatives thereof, such as glucosamine, galactosamine, acetylglucose, acetylgalactose, N-acetylglucosamine, N-acetyl-galactosamine, galactosyl-N-acetylglucosamine, N-acetylneuraminic acid (sialic acid), mycaminose, desosamine, L-vancosamine, 3-desmethyl-vancosamine, 3-epi-vancosamine, 4-epi-vancosamine, acosamine, 3-amino-glucose, 4deoxy-3-amino-glucose, actinosamine, daunosamine, 3-epi-daunosamine, ristosamine, N-methyl-D-glucamine, and the like; and aminomethyl and dimethylamino derivatives thereof.

Any one of the preceding processes wherein OR¹ is of the formula

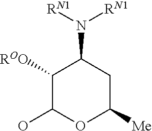

where each R^{N1} is independently selected in each instance from H and acyl, and alkyl, cycloalkyl, arylalkyl, and heteroarylalkyl, each of which is optionally substituted; and R^{O} is H or acyl, or alkyl, cycloalkyl, arylalkyl, and heteroarylalkyl, each of which is optionally substituted. In another embodiment, at least one R^{N1} is methyl. In another embodiment, both R^{N1} are methyl. In another embodiment, R^{O} is H or acyl. In another embodiment, R^{O} is H.

Any one of the preceding processes wherein R¹ is desosaminyl.

Any one of the preceding processes wherein R¹ is N-desmethyl desosaminyl.

A composition comprising solithromycin that is substantially free of or free of desfluoro solithromycin.

A composition comprising solithromycin that comprises less than about 1%, less than about 0.5%, less than about 0.4%, less than about 0.3%, less than about 0.2%, less than about 0.15%, less than about 0.1%, less than about 0.05%, or less than about 0.03% desfluoro solithromycin.

A composition comprising solithromycin that is substantially free of or free of N-desmethyl solithromycin.

A composition comprising solithromycin that comprises less than about 1%, less than about 0.5%, less than about 0.4%, less than about 0.3%, less than about 0.2%, less than about 0.15%, less than about 0.1%, less than about 0.05%, or less than about 0.03% N-desmethyl solithromycin.

Also described herein is a process for preparing solithromycin benzoate, or a salt thereof, where the process includes the step

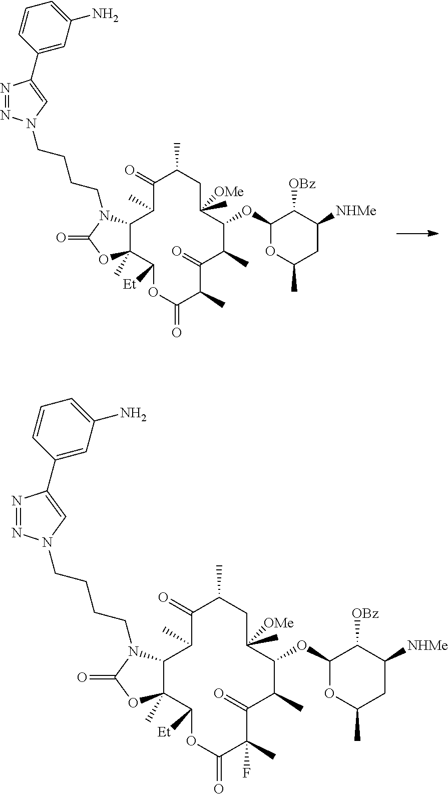

including salts of each of the foregoing.

Also described herein is a process for preparing solithromycin, or a salt thereof, where the process includes preparing a fluorinated compound described herein, and converting that fluorinated compound into solithromycin, or a salt thereof.

In each of the foregoing and each of the following embodiments, unless otherwise indicated, it is to be understood that the formulae include and represent not only all pharmaceutically acceptable salts of the compounds, but also include any and all hydrates and/or solvates of the compound formulae. It is appreciated that certain functional groups, such as the hydroxy, amino, and like groups form complexes and/or coordination compounds with water and/or various solvents, in the various physical forms of the compounds. Accordingly, the above formulae are to be understood to be a description of such hydrates and/or solvates, including pharmaceutically acceptable solvates.

In each of the foregoing and each of the following embodiments, unless otherwise indicated, it is also to be understood that the formulae include and represent any and all crystalline forms, partially crystalline forms, and non-crystalline and/or amorphous forms of the compounds.

In each of the foregoing and each of the following embodiments, unless otherwise indicated, it is also to be understood that the formulae include and represent each possible isomer, such as stereoisomers and geometric isomers, both individually and in any and all possible mixtures.

As used herein, the term “solvates” refers to compounds described herein complexed with a solvent molecule. It is appreciated that compounds described herein may form such complexes with solvents by simply mixing the compounds with a solvent, or dissolving the compounds in a solvent. It is appreciated that where the compounds are to be used as pharmaceuticals, such solvents are pharmaceutically acceptable solvents. It is further appreciated that where the compounds are to be used as pharmaceuticals, the relative amount of solvent that forms the solvate should be less than established guidelines for such pharmaceutical uses, such as less than International Conference on Harmonization (ICH) Guidelines. It is to be understood that the solvates may be isolated from excess solvent by evaporation, precipitation, and/or crystallization. In some embodiments, the solvates are amorphous, and in other embodiments, the solvates are crystalline.

It is to be understood that each of the foregoing embodiments may be combined in chemically relevant ways to generate subsets of the embodiments described herein. Accordingly, it is to be further understood that all such subsets are also illustrative embodiments of the invention described herein.

The compounds described herein may contain one or more chiral centers, or may otherwise be capable of existing as multiple stereoisomers. It is to be understood that in one embodiment, the invention described herein is not limited to any particular sterochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “alkyl” includes a chain of carbon atoms, which is optionally branched. As used herein, the terms “alkenyl” and “alkynyl” each include a chain of carbon atoms, which is optionally branched, and include at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₈, C₁-C₆, and C₁-C₄, and C₂-C₂₄, C₂-C₁₂, C₂-C₈, C₂-C₆, and C₂-C₄, and the like Illustratively, such particularly limited length alkyl groups, including C₁-C₈, C₁-C₆, and C₁-C₄, and C₂-C₈, C₂-C₆, and C₂-C₄, and the like may be referred to as lower alkyl. It is to be further understood that in certain embodiments alkenyl and/or alkynyl may each be advantageously of limited length, including C₂-C₂₄, C₂-C₁₂, C₂-C₈, C₂-C₆, and C₂-C₄, and C₃-C₂₄, C₃-C₁₂, C₃-C₈, C₃-C₆, and C₃-C₄, and the like Illustratively, such particularly limited length alkenyl and/or alkynyl groups, including C₂-C₈, C₂-C₆, and C₂-C₄, and C₃-C₈, C₃-C₆, and C₃-C₄, and the like may be referred to as lower alkenyl and/or alkynyl. It is appreciated herein that shorter alkyl, alkenyl, and/or alkynyl groups may add less lipophilicity to the compound and accordingly will have different pharmacokinetic behavior. In embodiments of the invention described herein, it is to be understood, in each case, that the recitation of alkyl refers to alkyl as defined herein, and optionally lower alkyl. In embodiments of the invention described herein, it is to be understood, in each case, that the recitation of alkenyl refers to alkenyl as defined herein, and optionally lower alkenyl. In embodiments of the invention described herein, it is to be understood, in each case, that the recitation of alkynyl refers to alkynyl as defined herein, and optionally lower alkynyl. Illustrative alkyl, alkenyl, and alkynyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like, and the corresponding groups containing one or more double and/or triple bonds, or a combination thereof.

As used herein, the term “alkylene” includes a divalent chain of carbon atoms, which is optionally branched. As used herein, the term “alkenylene” and “alkynylene” includes a divalent chain of carbon atoms, which is optionally branched, and includes at least one double bond or triple bond, respectively. It is to be understood that alkynylene may also include one or more double bonds. It is to be further understood that in certain embodiments, alkylene is advantageously of limited length, including C₁-C₂₄, C₁-C₁₂, C₁-C₈, C₁-C₆, and C₁-C₄, and C₂-C₂₄, C₂-C₁₂, C₂-C₈, C₂-C₆, and C₂-C₄, and the like. Illustratively, such particularly limited length alkylene groups, including C₁-C₈, C₁-C₆, and C₁-C₄, and C₂-C₈, C₂-C₆, and C₂-C₄, and the like may be referred to as lower alkylene. It is to be further understood that in certain embodiments alkenylene and/or alkynylene may each be advantageously of limited length, including C₂-C₂₄, C₂-C₁₂, C₂-C₈, C₂-C₆, and C₂-C₄, and C₃-C₂₄, C₃-C₁₂, C₃-C₈, C₃-C₆, and C₃- C₄, and the like. Illustratively, such particularly limited length alkenylene and/or alkynylene groups, including C₂-C₈, C₂-C₆, and C₂-C₄, and C₃-C₈, C₃-C₆, and C₃-C₄, and the like may be referred to as lower alkenylene and/or alkynylene. It is appreciated herein that shorter alkylene, alkenylene, and/or alkynylene groups may add less lipophilicity to the compound and accordingly will have different pharmacokinetic behavior. In embodiments of the invention described herein, it is to be understood, in each case, that the recitation of alkylene, alkenylene, and alkynylene refers to alkylene, alkenylene, and alkynylene as defined herein, and optionally lower alkylene, alkenylene, and alkynylene. Illustrative alkyl groups are, but not limited to, methylene, ethylene, n-propylene, isopropylene, n-butylene, isobutylene, sec-butylene, pentylene, 1,2-pentylene, 1,3-pentylene, hexylene, heptylene, octylene, and the like.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are each subsets of cycloalkenyl. It is to be understood that cycloalkyl may be polycyclic. Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e.

alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amino and derivatives thereof” includes amino as described herein, and alkylamino, alkenylamino, alkynylamino, heteroalkylamino, heteroalkenylamino, heteroalkynylamino, cycloalkylamino, cycloalkenylamino, cycloheteroalkylamino, cycloheteroalkenylamino, arylamino, arylalkylamino, arylalkenylamino, arylalkynylamino, heteroarylamino, heteroarylalkylamino, heteroarylalkenylamino, heteroarylalkynylamino, acylamino, and the like, each of which is optionally substituted. The term “amino derivative” also includes urea, carbamate, and the like.

As used herein, the term “hydroxy and derivatives thereof” includes OH, and alkyloxy, alkenyloxy, alkynyloxy, heteroalkyloxy, heteroalkenyloxy, heteroalkynyloxy, cycloalkyloxy, cycloalkenyloxy, cycloheteroalkyloxy, cycloheteroalkenyloxy, aryloxy, arylalkyloxy, arylalkenyloxy, arylalkynyloxy, heteroaryloxy, heteroarylalkyloxy, heteroarylalkenyloxy, heteroarylalkynyloxy, acyloxy, and the like, each of which is optionally substituted. The term “hydroxy derivative” also includes carbamate, and the like.

As used herein, the term “thio and derivatives thereof” includes SH, and alkylthio, alkenylthio, alkynylthio, heteroalkylthio, heteroalkenylthio, heteroalkynylthio, cycloalkylthio, cycloalkenylthio, cycloheteroalkylthio, cycloheteroalkenylthio, arylthio, arylalkylthio, arylalkenylthio, arylalkynylthio, heteroarylthio, heteroarylalkylthio, heteroarylalkenylthio, heteroarylalkynylthio, acylthio, and the like, each of which is optionally substituted. The term “thio derivative” also includes thiocarbamate, and the like.

As used herein, the term “acyl” includes formyl, and alkylcarbonyl, alkenylcarbonyl, alkynylcarbonyl, heteroalkylcarbonyl, heteroalkenylcarbonyl, heteroalkynylcarbonyl, cycloalkylcarbonyl, cycloalkenylcarbonyl, cycloheteroalkylcarbonyl, cycloheteroalkenylcarbonyl, arylcarbonyl, arylalkylcarbonyl, arylalkenylcarbonyl, arylalkynylcarbonyl, heteroarylcarbonyl, heteroarylalkylcarbonyl, heteroarylalkenylcarbonyl, heteroarylalkynylcarbonyl, acylcarbonyl, and the like, each of which is optionally substituted.

As used herein, the term “carbonyl and derivatives thereof” includes the group C(O), C(S), C(NH) and substituted amino derivatives thereof.

As used herein, the term “carboxylic acid and derivatives thereof” includes the group CO₂H and salts thereof, and esters and amides thereof, and CN.

As used herein, the term “sulfinic acid or a derivative thereof” includes SO₂H and salts thereof, and esters and amides thereof.

As used herein, the term “sulfonic acid or a derivative thereof” includes SO₃H and salts thereof, and esters and amides thereof.

As used herein, the term “sulfonyl” includes alkylsulfonyl, alkenylsulfonyl, alkynylsulfonyl, heteroalkylsulfonyl, heteroalkenylsulfonyl, heteroalkynylsulfonyl, cycloalkylsulfonyl, cycloalkenylsulfonyl, cycloheteroalkylsulfonyl, cycloheteroalkenylsulfonyl, arylsulfonyl, arylalkylsulfonyl, arylalkenylsulfonyl, arylalkynylsulfonyl, heteroarylsulfonyl, heteroarylalkylsulfonyl, heteroarylalkenylsulfonyl, heteroarylalkynylsulfonyl, acylsulfonyl, and the like, each of which is optionally substituted.

As used herein, the term “phosphinic acid or a derivative thereof” includes P(R)O₂H and salts thereof, and esters and amides thereof, where R is alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, or heteroarylalkyl, each of which is optionally substituted.

As used herein, the term “phosphonic acid or a derivative thereof” includes PO₃H₂ and salts thereof, and esters and amides thereof.

As used herein, the term “hydroxylamino and derivatives thereof” includes NHOH, and alkyloxylNH alkenyloxylNH alkynyloxylNH heteroalkyloxylNH heteroalkenyloxylNH heteroalkynyloxylNH cycloalkyloxylNH cycloalkenyloxylNH cycloheteroalkyloxylNH cycloheteroalkenyloxylNH aryloxylNH arylalkyloxylNH arylalkenyloxylNH arylalkynyloxylNH heteroaryloxylNH heteroarylalkyloxylNH heteroarylalkenyloxylNH heteroarylalkynyloxylNH acyloxy, and the like, each of which is optionally substituted.

As used herein, the term “hydrazino and derivatives thereof” includes alkylNHNH, alkenylNHNH, alkynylNHNH, heteroalkylNHNH, heteroalkenylNHNH, heteroalkynylNHNH, cycloalkylNHNH, cycloalkenylNHNH, cycloheteroalkylNHNH, cycloheteroalkenylNHNH, arylNHNH, arylalkylNHNH, arylalkenylNHNH, arylalkynylNHNH, heteroarylNHNH, heteroarylalkylNHNH, heteroarylalkenylNHNH, heteroarylalkynylNHNH, acylNHNH, and the like, each of which is optionally substituted.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups, also referred to herein as aryl substituents or heteroaryl substituents, respectively, illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical —(CH₂)ₓZ^{X}, where x is an integer from 0-6 and Z^{X} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N-(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N-(C₁-C₆ alkyl)alkylcarbonylaminoalkyl, cyano, and nitro; or Z^{X} is selected from —CO₂R⁴ and —CONR⁵R⁶, where R⁴, R⁵, and R⁶ are each independently selected in each occurrence from hydrogen, C₁-C₆ alkyl, aryl-C₁-C₆ alkyl, and heteroaryl-C₁-C₆ alkyl.

The term “protecting group” as used herein general refers to any radical that is reversibly bonded to a functional group and is used to block or partially block the reactivity of that functional group to a predetermined set of conditions, such as reaction conditions. Illustratively, nitrogen protecting groups are reversibly bonded to amines to block or partially block the reactivity of the amine under a predetermined set of conditions. Illustrative nitrogen protecting groups include, but are not limited to, carbamates, such as t-Boc, Fmoc, and the like.

As used herein, the term “leaving group” refers to a reactive functional group that generates an electrophilic site on the atom to which it is attached such that nucleophiles may be added to the electrophilic site on the atom. Illustrative leaving groups include, but are not limited to, halogens, optionally substituted phenols, acyloxy groups, sulfonoxy groups, and the like. It is to be understood that such leaving groups may be on alkyl, acyl, and the like. Such leaving groups may also be referred to herein as activating groups, such as when the leaving group is present on acyl. In addition, conventional peptide, amide, and ester coupling agents, such as but not limited to PyBop, BOP-Cl, BOP, pentafluorophenol, isobutylchloroformate, and the like, form various intermediates that include a leaving group, as defined herein, on a carbonyl group.

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

As used herein, the terms “treating”, “contacting” or “reacting” when referring to a chemical reaction generally mean to add or mix two or more reagents under appropriate conditions that allows a chemical transformation or chemical reaction to take place, and/or to produce the indicated and/or the desired product. It is to be understood that the reaction which produces the indicated and/or the desired product may not necessarily result directly from the combination of two reagents which were initially added. In other words, there may be one or more intermediates which are produced in the mixture which ultimately leads to the formation of the indicated and/or the desired product.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. In addition, it is to be understood that the compositions may be prepared from various co-crystals of the compounds described herein.

Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

In addition to the illustrative dosages and dosing protocols described herein, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

The term “administering” as used herein includes all means of introducing the compounds and compositions described herein to the host animal, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and/or vehicles.

Illustrative formats for oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

The following examples further illustrate specific embodiments of the invention; however, the following illustrative examples should not be interpreted in any way to limit the invention.

### EXAMPLES

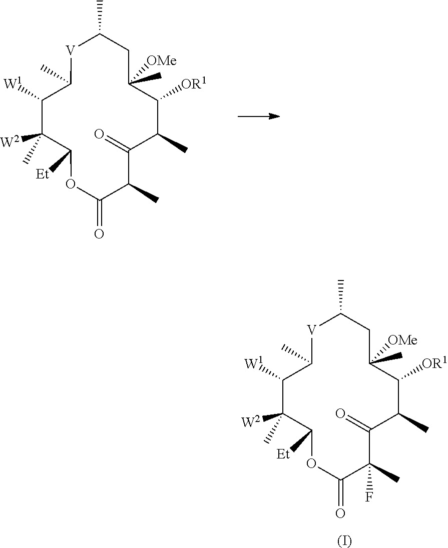

### Example

General procedure for preparing fluoroketolides. A solution of starting material is cooled to a temperature in the range from about −15° C. to about −40° C. An amine base described herein (2-3 eq) is added. A fluorinating reagent (1-2 eq), or solution of a fluorinating agent is added. After acceptable or complete conversion, the reaction is quenched with water. The compound of formula (I) is isolated from the organic layer, and optionally precipitated from an alcohol/water mixture.

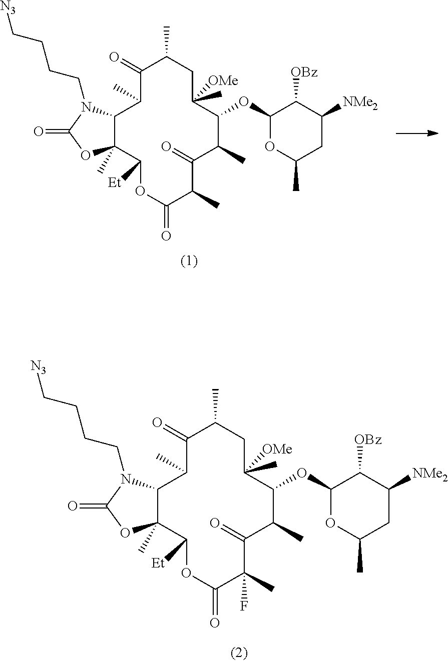

### Example

(11-N-(4-azido-butyl)-5-(2′-benzoyl-desosaminyl)-3-oxo-2-fluoro-6-O-methyl-erythronolide A, 11,12-cyclic carbamate) (CEM-276, compound (2)). CEM-275 (compound (1), 1.0 eq) is added to DMF, isopropyl acetate, or a mixture of DMF/isopropyl acetate (2-10 volumes) and stirred at ambient temperature to give a clear solution. It is to be understood that the foregoing concentrations are not critical. The solution is cooled to and maintained at −20° C. to −30° C. with stirring. DBU (2-3 eq) is added, then a solution of NFSI (1.1-1.5 eq) in DMF, isopropyl acetate, or a mixture of DMF/isopropyl acetate (1-3 volumes) is added. The mixture is stirred until acceptable or complete conversion is observed, such as by TLC, HPLC, and the like. Isopropyl acetate (2-7 volumes) and chilled water (2-10 volumes) are added, optionally in stages. The organic layer is removed, and the aqueous layer is extracted with isopropyl acetate. The combined organic layers are washed with water. Formaldehyde (0.1-0.3 eq) and formic acid (0.5-1.0 eq) are added to the solution at ambient temperature, then the mixture is heated to 45-50° C. until acceptable or complete conversion is observed, such as by TLC, HPLC, and the like. The solution is cooled to ambient temperature, water is added, and the pH is adjusted to 7-8 with aqueous ammonia. The aqueous layer is removed, and the organic layer is washed with water. The organic layer is concentrated under vacuum. Isopropanol (IPA) is added and the mixture is heated. Water is added, and the resulting slurry is cooled to ambient temperature, and filtered. The resulting solid is washed with water and dried under vacuum to give CEM-276.

### Example

The general process described herein was performed on a scale of 50 g (2 independent runs) followed by in situ methylation to provide (2) in 90-92% isolated yield with 98% HPLC purity, with (1) in the range of about 0.05-0.1%. Neither (1-DM) nor (2-DM) were detected.

### Example

The general process described herein was performed on a commercial scale of 100 g (3 independent runs) followed by in situ methylation to provide (2) in a 89-90% isolated yield with 98-99% HPLC purity, with (1) in the range of about 0.07-0.18%. Neither (1-DM) nor (2-DM) were detected.

### Example

The general process described herein was performed on a commercial scale of 200 g (2 independent runs) followed by in situ methylation to provide (2) in a consistent 88% isolated yield with 98-99% HPLC purity, with (1) consistently at 0.07%, and (1-DM) and (2-DM) consistently not detectable.

### Example

The general process described herein was performed on a commercial scale of 3 kg followed by in situ methylation to provide (2) in 93% isolated yield with 98.4% HPLC purity.

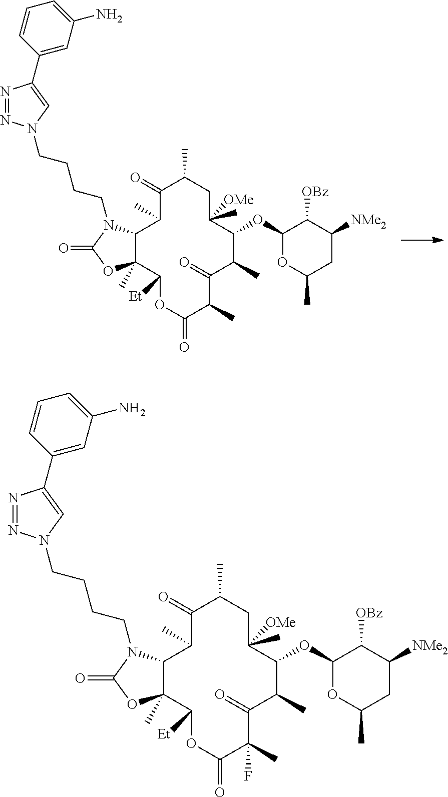

### Example

11-N-(3-amino-phenyl-1-yl-[1,2,3]-triazole-1-yl]butyl)-5-(2′-benzoyldesosaminyl)-3-oxo-2-fluoro-erythronolide A, 11,12-cyclic carbamate. 11-N-(3-amino-phenyl-1-yl-[1,2,3]-triazole-1-yl]butyl)-5-(2′-benzoyldesosaminyl)-3-oxo-erythronolide A, 11,12-cyclic carbamate was prepared according to WO 2009/055557 from (1) and 3-ethynylphenylamine. The general process described herein was performed to provide a 97% conversion, 90% isolated yield of the title compound, only 0.36% remaining unfluorinated starting material, and 2.3% N-desmethyl before in situ methylation.

### Example

11-N-(3-amino-phenyl-1-yl-[1,2,3]-triazole-1-yl]butyl)-5-(2′-benzoyldesosaminyl)-3-oxo-2-fluoro-erythronolide A, 11,12-cyclic carbamate. 11-N-(4-azidobutyl)-5-(2′-benzoyldesosaminyl)-3-oxo-2-fluoro-6-O-methylerythronolide A, 11,12-cyclic carbamate, 3-ethynylphenylamine, copper iodide, and diisopropylethylamine are reacted in acetonitrile as described in WO 2009/055557 to prepare 11-N-(3-amino-phenyl-1-yl-[1,2,3]-triazole-1-yl]butyl)-5-(2′-benzoyldesosaminyl)-3-oxo-2-fluoro-erythronolide A, 11,12-cyclic carbamate.

### Example

Solithromycin. 11-N-(3-amino-phenyl-1-yl-[1,2,3]-triazole-1-yl]butyl)-5-(2′-benzoyldesosaminyl)-3-oxo-2-fluoro-erythronolide A, 11,12-cyclic carbamate is dissolved in methanol and heated at reflux, as described in WO 2009/055557 to prepare solithromycin.

### Comparative Example

A process for preparing (2) from (1) is disclosed in WO 2009/055557. The process was performed as described on a scale of 10 g (2 independent runs) to provide a 65% yield of (2) having 89% HPLC purity, and contaminated with 9.9% unreacted starting material (1).

### Example

The foregoing process was adapted by using NFSI and lithium tert-butoxide as the base. Conversion to (2) was incomplete with 9-11% remaining (1).

### Comparative Example

The process disclosed in WO 2009/055557 was modified by using potassium pentoxide as the base. Conversion to (2) was very low or not observed. In addition, one or more unknown side products was formed.

### Comparative Example

The process disclosed in WO 2009/055557 was modified by using lithium tert-butoxide as the base. Conversion to (2) was very low with 9-11% unreacted (1) remaining. In addition, unknown side products was also formed.

### Comparative Example

The process disclosed in WO 2009/055557 was modified by using NaH as the base. Conversion to (2) was very low with significant decomposition to unknown side products.

### Comparative Example

The process disclosed in WO 2009/055557 was modified by using Selectfluor as the fluorinating agent. Conversion to (2) was comparable with 29% unreacted (1) remaining.

### Comparative Example

The process disclosed in WO 2009/055557 was modified by using NaHMDS as the base. Conversion to (2) was very low with significant decomposition to unknown side products.

### Comparative Example

The process disclosed in WO 2009/055557 was modified by using K₂CO₃ as the base. Conversion to (2) was not observed. Instead, significant decomposition to one or more unknown side products was observed.

### Comparative Example

The process disclosed in WO 2009/055557 was modified by using K₂CO₃ as the base in toluene/water with tetra-n-butylammonium bromide (TBAB) phase transfer catalyst. Conversion to (2) was not observed. In addition, one or more unknown side products was formed.

### Comparative Example

The process disclosed in WO 2009/055557 was modified by using NFSI or Selectfluor and a Lewis Acid or transition metal catalyst, such as MgClO₄, Ti(iOPR)₄, Pd(OAc)₂, and the like, in place of the base. Conversion to (2) was not observed. In addition, one or more unknown side products was formed.

### Comparative Example

The process disclosed in WO 2009/055557 was modified by using DMF as the solvent. Conversion to (2) was low with 24% unreacted (1) remaining.

### Comparative Example

The process disclosed in WO 2009/055557 was modified by using 1:1 THF/DCM as the solvent. Conversion to (2) was low with 12-15% unreacted (1) remaining. In addition, unknown side products was also formed.

Each publications cited herein is incorporated herein by reference.

1. A process for preparing a fluoroketolide of formula (I) 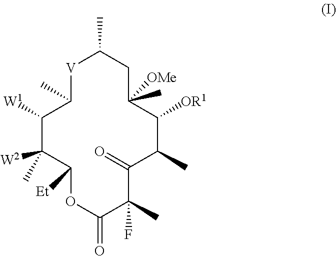
the process comprising contacting a compound of the formula 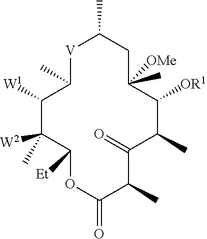
with a fluorinating agent and an amine base; wherein
R¹ is H or acyl, or le is a monosaccharide, such as methylamino or dimethylamino containing monosaccharide;
V is CH₂—N(R), C═Q or C═NQ¹; where Q is O or (NR, H); where R is hydrogen or optionally substituted alkyl; and Q¹ is hydroxy or a derivative thereof or amino or a derivative thereof; and
W¹ is hydroxy or a derivative thereof; and W² is H, or hydroxy or a derivative thereof; or W¹ and W² are taken together with the attached carbon atoms to form an oxygen and/or nitrogen containing heterocycle, each of which is optionally substituted.

2. The process of claim 1 wherein the base is a cyclic amine base, a non-aromatic amine base, or an amine base with a conjugate acid pKa of at least about 11, or a combination of the foregoing.

3. The process of claim 1 wherein the base is sterically hindered.

4. The process of claim 1 wherein the base is a diamine.

5. The process of claim 1 wherein the base includes at least one nitrogen that does not have a hydrogen.

6. The process of claim 1 wherein the base includes at least one C=N group.

7. The process of claim 1 wherein the base is DBN or DBU, or a combination thereof.

8. The process of claim 1 wherein the fluorinating agent is selected from the group consisting of NFSi, Selectfluor, and F-TEDA, and combinations thereof

9. The process of claim 1 wherein the compound of formula (I) is 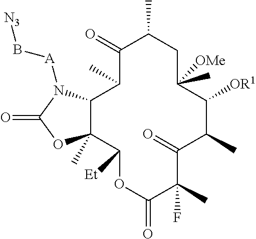
or a salt thereof.

10. The process of claim 1 wherein the compound of formula (I) is 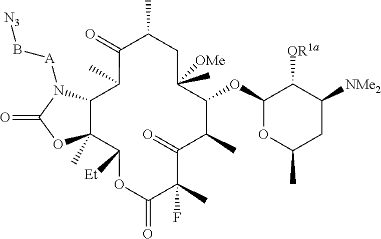
or a salt thereof.

11. The process of claim 1 wherein the compound of formula (I) is solithromycin or a salt thereof.

12. The process of claim 1 wherein the starting compound is of the formula 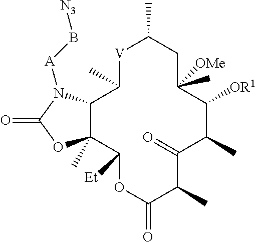
or a salt thereof.

13. The process of claim 1 claim 8 wherein the starting compound is of the formula 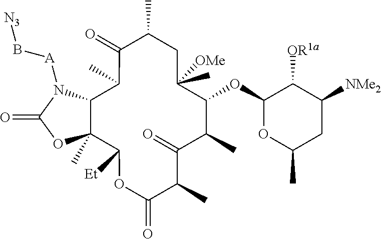
or a salt thereof.

14. A process for preparing a compound of formula (I), the process comprising or further comprising contacting a compound of the formula (DM) 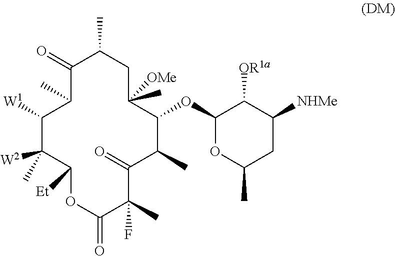
or a salt thereof, with a methylating agent; wherein:
R^{1a} is H or acyl;
V is CH₂—N(R), C═Q or C═NQ¹; where Q is O or (NR, H); where R is hydrogen or optionally substituted alkyl; and Q¹ is hydroxy or a derivative thereof or amino or a derivative thereof;
W¹ is hydroxy or a derivative thereof; and W² is H, or hydroxy or a derivative thereof; or W¹ and W² are taken together with the attached carbon atoms to form an oxygen and/or nitrogen containing heterocycle, each of which is optionally substituted.

15. The process of claim 14 wherein the compound of formula (DM) is 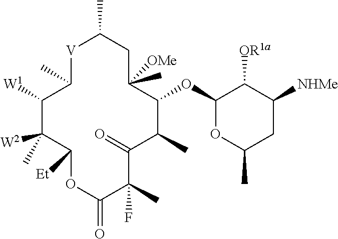
or a salt thereof.

16. The process of claim 14 wherein the compound of formula (DM) is 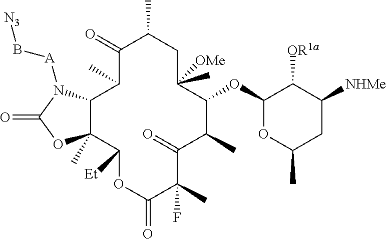
or a salt thereof.

17. A composition comprising solithromycin that is substantially free of desfluoro solithromycin or N-desmethyl solithromycin, or both.

18. The composition of claim 17 wherein the composition is substantially free of N desmethyl solithromycin.

19. The composition of claim 17 wherein the composition is substantially free of desfluoro solithromycin.
